(19) United States
(12) Patent Application Publication
(10) Pub. No.: US 2009/0221004 A1
(43) Pub. Date: Sep. 3, 2009

(54) Caspase-cleavage anti-keratin antibodies for detection of apoptosis

(76) Inventor: Anita Lim Hong, Cupertino, CA (US)

Correspondence Address: 212 Consulting, Inc., 851 Moraga Road, Bungalow B, Lafayette, CA, 94549, US

(21) Appl. No.: 12/154,846

(22) Filed: May 27, 2008

Related U.S. Application Data
(60) Provisional application No. 60/932,691, filed on Jun. 1, 2007.

(51) Int. Cl. G01N 33/574; C07K 16/40; G01N 33/53; G01N 33/00

(52) U.S. Cl. 435/7.23; 530/388.1; 530/389.1; 435/7.1; 435/7.92

(57) ABSTRACT

The present invention relates to the field of detecting and quantifying apoptosis. In one aspect, the invention is directed to an isolated monoclonal or polyclonal antibody that specifically recognizes caspase-generated products of K18 or K19 but does not react with intact K18 or K19. The antibody typically reacts with the cleavage site of K18 or K19 at Asp237. The binds to the epitope common to caspase-cleaved K18 and K19-(T/S)VEVD- and the Asp-proximal valine is essential for antibody recognition.

### PRIORITY CLAIM

This application claims priority to U.S. Provisional Patent Appl. No. 60/932,691 under 35 U.S.C. §119. Provisional application 60/932,691 is incorporated-by-reference into this document for all purposes.

### STATEMENT REGARDING FEDERALLY SPONSORED RESEARCH OR DEVELOPMENT

This work was supported by NIH grant DK47918, a VA Merit award and by NIH Digestive Disease Center grant DK56339.

### FIELD OF THE INVENTION

The invention relates to the field of detecting and quantifying apoptosis. More particularly, the present invention relates to antibodies that specifically recognize the newly exposed Asp237 residue at the carboxyl terminus resulting from caspase-cleavage at L1-2 linker region of K18/K19 during apoptosis, and to the utility of the antibodies in detecting apoptosis in tissues/cells undergoing apoptosis or in human and animal body fluids.

### BACKGROUND OF THE INVENTION

Intermediate filaments (IFs), together with actin microfilaments and tubulin microtubules, are one of three major cytoskeletal networks that are found in most eukaryotic cells (Ku et al., 1999). In contrast to microfilaments and microtubules, IFs are more diverse and are expressed in a tissue- and differentiation-specific manner (Fuchs and Cleveland 1998; Coulombe and Wong, 2004). IFs share a common structure that contains a central α-helical rod domain, which is flanked by non-α-helical amino-terminal and carboxy-terminal regions termed the “head” and “tail” domains, respectively (Steinert and Roop, 1988; Fuchs and Weber, 1994).

The coiled-coil α-helical rod domain is interrupted by short 8-17 amino acid stretches termed linkers (L) L1 (within rod subdomain I), L12 (separating rod subdomains 1 and 2) and L2 (within rod subdomain 2) (Herrman and Aebi, 2004). The head and tail domains contain the sites of several post-translational modifications, including phosphorylation and glycosylation, that play numerous regulatory roles (Omary et al., 2006; Pallari and Eriksson, 2006).

In epithelial cells, cytoplasmic IFs are composed of type I (K9-K20) and type II (K1-K8) keratins (Moll et al 1982; Coulombe and Omary, 2002; Schweizer et al., 2006). All epithelial cells express at least one type I and one type II keratin, and the two types of keratins exist as obligate non-covalent heteropolymers. For example, K8 and 18 heterodimers are abundantly co-expressed in the liver and other “simple” single-layered glandular epithelia and form heterodimers that may include variable levels of K7, K19 and K20 depending on the cell type (Ku et al 1999; Zhou et al, 2003). Keratin networks are highly dynamic and reorganize during differentiation, mitosis and apoptosis (Omary et al., 2006).

Although many of the keratin functions remain to be established, K8/K18 play important cytoprotective and structural support roles (Omary et al., 2002; Omary et al. 2004). For example, K8 knockout mice die on embryonic day 12 in the C57/BI strain (Baribault et al., 1993), but backcrossing to the FVB strain leads to survival with normal life-span survival in 50% of the animals in association with colorectal hyperplasia and inflammation (Baribault et al., 1994; Toivola et al., 2004; Habtezion et al., 2005).

Apoptosis is an important regulated form of cell death in normal and diseased tissues, and involves the activation of the proteolytic caspase cascade that is responsible for stepwise cleavage of many critical cellular proteins (Steller, 1995; Bredesen et al., 2006). Among cytoskeletal-related caspase substrate proteins (Timmer and Salvesen, 2007), K18 (Ku et al., 1997; Caulin et al.; 1997; Ku et al., 2001) and K19 (Ku et al., 1997) undergo caspase-mediated proteolysis during apoptosis at a highly conserved Asp237 within the L12 segment of the rod domain. Many other IF proteins also contain the conserved L12 caspase consensus sequence (Ku et al., 1997 and 2001; Caulin et al., 1997; Leers et al., 1999; Oshima, 2002; Marceau et al., 2007 in press), although some differences are found in residues at the N-terminal side in proximity to the highly conserved aspartate (Table 1). K18 is unique in that it includes a second caspase-cleavage site at Asp396 of the tail domain that is not conserved among other IF proteins (Caulin et al., 1997; Leers et al., 1999; Ku and Omary, 2001). Procaspases 3 and 9 specifically target K18 by the death effector domain containing DNA binding protein (DEDD) (Lee et al., 2002 and Dinsdale et al., 2004).

Caspase cleavage at K18 Asp396 is an early event in the apoptotic cascade, preceding annexin V reactivity, positive DNA fragmentation labeling, and Asp237 cleavage (Leers et al., 1999; Ku and Omary 2001). Caspase cleavage at Asp396 generates a neo-epitope that is specifically recognized by the M30 “CytoDeath” monoclonal antibody (Leers et al., 1999). The commercially available M30 antibody has been widely used for the detection of apoptosis in cultured cells (Schutte et al., 2004, Leer et al., 1999), tissues (Leers et al., 1999 and Grassi et al., 2004) and sera of patients with cancer (Linder et al., 2004; Kramer et al., 2006), chronic hepatitis C (Bantel et al., 2004; Volkmann et al., 2006) or nonalcoholic fatty liver disease (Wieckowska et al., 2006).

No reagents are available, however, that can detect K18 Asp237 caspase cleavage at Asp237 and the stability of the N-terminal Asp237-containing versus the C-terminal Asp396-containing fragment is unknown.

### SUMMARY OF THE INVENTION

The invention relates to the field of detecting and quantifying apoptosis.

In one aspect, the invention is directed to an isolated monoclonal or polyclonal antibody that specifically recognizes caspase-generated products of K18 or K19 but does not react with intact K18 or K19. The antibody typically reacts with the cleavage site of K18 or K19 at Asp237. The binds to the epitope common to caspase-cleaved K18 and K19-(T/S)VEVD- and the Asp-proximal valine is essential for antibody recognition.

In another aspect, the invention is directed to a method of detecting and/or quantifying the amount of caspase activation in vitro, in tissue samples, or in cells and tissues, where the method includes the steps of: contacting one or more of the monoclonal or polyclonal antibodies described in the preceding paragraph with a serum sample, a cellular sample or a tissue sample; and detecting the presence and/or quantifying the amount of caspase-generated K18 or K19 products in the sample.

In another aspect, the invention is directed to a method for performing sandwich ELISA. Steps for the method include: a plate is coated with one or more anti-K18 or anti-K19 antibodies to epitopes between the first amino acid to the Asp237 residue; the plate is washed with a blocking agent (e.g., BSA); a test sample (e.g., human or animal serum) is incubated on the plate; the plate is washed and incubated detect antibodies that react with cleavage sites of K18 or K19 at Asp237; the plate is washed and incubated with a secondary antibody conjugated to a labeling unit (e.g., enzyme that provides signal upon addition of substrate).

In another aspect, the invention is directed to a method of performing flow cytometry. Steps for the method include: permeabilizing one or more cells to allow entry by one or more antibodies; incubating the cells with one or more first antibodies that react with cleavage sites of K18 or K19 at Asp237; incubating the cells with a second antibody that contains a fluorescent marker is immunoreactive to the first; washing the cells; subjecting the cells to flow cytometry, where an increase in fluorescence intensity of a cell over control, non-apoptotic cells indicates the presence of apoptotic cells.

In another aspect, the invention is directed to a method of performing immunoblot analysis. Steps for the method include: subjecting a protein sample to SDS-PAGE at such conditions to yield an appropriate separation of proteins within the sample; transferring the proteins to a membrane; submersing the membrane in a blocking solution and contacting it with one or more first antibodies that react with cleavage sites of K18 or K19 at Asp237; incubating the cells with a second antibody that contains a fluorescent marker is immunoreactive to the first where the second antibody is conjugated to a labeling unit.

In another aspect, the invention is directed to a method of determining whether a compound, or mixture of compounds, induces apoptosis in a sample. The method includes the steps of: adding one or more compounds to a sample that contains K18 or K19; adding one or more antibodies that react with cleavage sites of K18 or K19 at Asp237; detecting binding between the antibodies and the cleavage products of K18 or K19.

In another aspect, the invention is directed to a method of determining whether a compound, or mixture of compounds, inhibits apoptosis in a sample. The method includes the steps of: adding one or more first compounds to a sample that contains K18 or K19; adding a second compound known to induce apoptosis in samples; adding one or more antibodies that react with cleavage sites of K18 or K19 at Asp237; detecting binding between the antibodies and the cleavage products of K18 or K19.

In another aspect, the invention is directed to a method for detecting acute injury. The method includes the steps of: contacting one or more antibodies that react with cleavage sites of K18 or K19 at Asp237 with a sample taken from a human or animal; detecting binding between the antibodies and cleavage products of K18 or K19.

In another aspect, the invention is directed to a method for detecting a chronic disease. The method includes the steps of: contacting one or more antibodies that react with cleavage sites of K18 or K19 at Asp237 with a sample taken from a human or animal; detecting binding between the antibodies and cleavage products of K18 or K19.

In another aspect, the invention is directed to a method for detecting cancer. The method includes the steps of: contacting one or more antibodies that react with cleavage sites of K18 or K19 at Asp237 with a sample taken from a human or animal; detecting binding between the antibodies and cleavage products of K18 or K19.

### BRIEF DESCRIPTION OF THE FIGURES

FIG. 1. Shows a schematic diagram of the caspase cleavage site in K18. The illustrated polypeptide is the intact K18 protein.

FIG. 2. Shows photographs of Western blot analysis of normal and apoptotic cells probed with antibodies specific to the K18/K19 apoptotic fragments.

FIG. 3. Shows photographs of indirect immunofluorescence staining of apoptotic cells probed with antibodies specific to the K18/K19 apoptotic fragments.

FIG. 4. The Asp237 antibody specifically recognizes caspase-generated but not intact K18 in cultured human cells and mouse liver. A) Schematic of caspase-mediated sequential digestion of K18 at Asp396 then Asp237 during apoptosis. The diagram also shows the prototype IF structure of the “Head”, “Rod” and “Tail” domains. The α-helical rod domain includes subdomains 1A, IB and II which are separated by the linker L1 and L12 non-α-helical short stretches. B) The anti-K18 Asp237 was pre-incubated with or without the immunization peptide (0.5 mg/ml, 4° C., 16 h), then used to immunoblot lysates of HT29 cells. The cell lysates were generated after culturing cells in the presence of anisomycin (An) to induce apoptosis or DMSO (vehicle control). C) Apoptosis was induced in HT29 cells, as in panel B using An, or in mouse livers by intraperitoneal injection of Fas antibody. The cell or tissue lysates were separated on SDS-PAGE, followed by blotting using an antibody that recognizes intact (i) K18 or the K18 Asp237 Ab that was purified using a peptide column (as described in Experiments and Results). Note that the Asp237 Ab recognizes only the caspase-cleaved fragment generated during apoptosis but not intact K18.

FIG. 5. Asp237 antibodies bind to VEVD K18 and K19 sequences but not to the VEMV K14 sequence. A) Purified recombinant human K14 (lanes 1 and 2) and K18 (lanes 3 and 4) were incubated with or without caspase-3 (Casp 3) (37° C., 2 h), then separated using SDS-PAGE followed by Coomassie staining or blotting with the K18 Asp237 Ab. Arrowhead indicates cleaved K14 seen in lane 2. Although only a small portion of K18 was cleaved by caspase 3 (cleaved K18 fragments were difficult to visualize by Coomassie staining), the Asp237 Ab readily detects the K18 caspase-generated fragment. B) BHK cells (which do not express endogenous keratins) were transfected with K18 or K19 cDNA (together with K8 eDNA). After 2 d, anisomycin (An) was added followed 24 h later by analysis of the cell lysates by immunoblotting using antibodies specific to the indicated antigens. Note that both the K18 Asp237 (K18 D237) and K19 Asp237 (K19 D237) antibodies bind to the 29 kD fragments derived from either intact K18 or K19.

FIG. 6. Immunofluorescence staining of apoptotic cells using the K18 Asp237 antibody. A) HT29 cells were treated with An or DMSO (vehicle control) for 20 h. Cells were then washed, fixed with methanol (−20° C., 5 min) followed by triple staining with the indicated antibodies or Toto-3 (for nuclear staining). B) The boxed area in Panel A-h is displayed as enlargement of each triple stain component. Scale bars are 50 μm (in panel e) for all panels in A, and 10 μm (in red channel) for all panels in B.

FIG. 7. The K18 Asp237 antibody stains both filaments and aggregates in apoptotic cells. A) HT29 cells were treated with An for 4 h (“Early”) or 16 h (“Late”) followed by double staining using Toto-3 (nuclear staining) or the anti-Asp237 Ab. At the early stage of apoptosis, Asp237 Ab stains primarily filaments which subsequently reorganize into aggregates in association with nuclear fragmentation. B) The cells used in Panel A were double stained using the anti-Asp396 (M30) and Asp237 K18 antibodies. Note in panel B-a that the cell highlighted by an asterisk and the intracellular area indicated by an arrowhead were only stained by Asp396 but not Asp237. Note in Panels B-d,e,f that many dots stain exclusively with the Asp237 Ab. All scale bars (in A and B) are 10 μm.

FIG. 8. Detection of Fas-mediated apoptosis in mouse liver using the K18 Asp237 antibody. Mice were injected intraperitoneally with Fas-antibody followed by isolation of the livers for immunofluorescence staining as described in Experiments and Results. Normal and injured livers were triple stained with the indicated markers. Panels A and B display low and high magnification images, respectively. N: nucleus; CV: central vein; asterisks (panel A-d) exemplify areas of cell drop-off; dotted area (panel A-e) highlights an area of injury and cell apoptosis. Scale bars are 50 μm (A-e) for panels a-f in A, and 10 μm (B-e) for panels a-f in B.

FIG. 9. Immunofluorescence staining of apoptotic cells in human cirrhotic livers using the K18 Asp237 antibody. Alcohol and hepatitis B associated cirrhotic livers were double stained using antibodies to K18 Asp237 (a,c) and total K18 (b,d). Asterisks in panels a-d highlight cells that are negative for Asp237 but positive for K18 staining. The hepatitis B liver was also double stained with antibodies to Asp237/396 (e,f) which showed a similar pattern with the two antibodies. Scale bar is 10 μm (in panel e) for all images.

FIG. 10. Detection of apoptosis in mouse and human tissues using K18 Asp237 immunohistochemical staining. Formalin-fixed and paraffin-embedded Fas-treated mouse liver (a-d); and human esophageal, gastric and colonic cancer tissues (e-h) were stained with: anti-Asp237 Ab (a, e, f, g), anti-total K18 Ab (c, h), preimmune normal rabbit serum (b), or hemtoxylin and eosin (H&E) (d). The arrows in panel d indicate cells with typical condensed or fragmented nuclei. Note the selective staining of apoptotic cells (brown color in panels e, f and g) using the Asp237 Ab. Scale bars are 50 μm (in panel b) for images a-d, 50 μm (in panel h) for images e,f,h, and 25 μm (in panel g).

FIG. 11. Detection of caspase-generated Asp237 keratin fragments in cell culture supernatants, and in human and mouse serum. The K18 Asp237 Ab was used in a sandwich ELISA to measure caspase-generated fragments that are released into: (a) the culture media of HT29 cells (+anisomycin), (b) sera of mice±Fas Ab injection to induce hepatocyte apoptosis, or (c) sera of patients with cirrhosis or controls. The representative wells of ELISA plates, the statistical data, and the number of samples analyzed for each control or apoptosis group are also shown.

FIG. 12. Detection of caspase-cleaved K18 fragment by immunoblotting using the Asp237 Ab. Induction of apoptosis in cultured cells and mouse liver is as described in Experiments and Results. A) After centrifugation (18,000 g; 15 min) of culture media, equal amount of supernatants were subjected to SDS-PAGE followed by blotting using the Asp237 Ab. B) Sera of mice (+/−Fas Ab ip injection to induce hepatocyte apoptosis) were collected and analyzed by immunoblotting as in panel A. C) A keratin-enriched fraction was isolated using a high salt extraction method (1) from human cirrhotic and normal livers followed by analysis by immunoblotting. See Ku N O, Toivola D M, Zhou Q, Tao G Z, Zhong B, Omary M B: Studying simple epithelial keratins in cells and tissues. Methods Cell Biol. 2004, 78:489-517.

### DESCRIPTION OF THE INVENTION

The present invention relates to an antibody specifically recognizing the caspase (e.g., caspase-3 or -6)-generated products of K18 and K19 but not reacting with intact proteins. To detect the activity of caspases, the inventors have produced an antibody reacting with the cleavage site of K18 and K19 at Asp237. Previous studies have shown that K18/K19 undergo caspase cleavage at the conserved Asp237 in L1-2 linker region of their rod domains during apoptosis (Ku et al. 1997; Caulin et al., 1997). Oligopeptides with an amino acid sequence corresponding to the N-terminal side of the cleaved aspartic acid residue are chemically synthesized and used for immunizing rabbits. After several immunizations, anti-serum is obtained.

An antibody that binds strongly to the antigen oligopeptide is purified from the anti-sera. The purified antibody does not bind to the intact K18/K19 proteins, but specifically binds to the product cleaved by caspase-3 or -6. By using this antibody in the Western blot analysis and immunostaining of cells and tissues, the detection of caspase activation becomes possible through cleavage of K18/K19 in vitro, in tissue samples, or in cells and tissues.

The term “antibody” according to the present invention means the whole antibody molecule or its fragments, which can bind to the cleavage product of K18 and K19 as an antigen. The antibodies described in this invention may be polyclonal or monoclonal. Antibodies according to the present invention can be produced by various methods known to those of skill in the art.

The inventors hypothesized that generation of immunologic reagents for K18/K19 Asp237 detection could provide a powerful tool to characterize its potential dynamic nature and serve as a marker to monitor keratin caspase digestion during apoptosis in situ. The inventors demonstrated these antibodies are highly versatile and sensitive in monitoring human and mouse K18/K19 digestion during apoptosis. The Asp237-containing K18/K19 fragment is highly stable and can be detected in mouse and human serum which indicates that it is a useful marker for monitoring apoptosis in several human and parallel mouse disease models.

Unique features of the Asp237 antibody: Apoptosis involves many physiologic and pathologic human contexts, with an ordered activation of the proteolytic caspase cascade that results in stepwise cleavage of many cellular proteins (Steller, 1995; Bredesen et al., 2006) including keratin 18 and 19 (Caulin et al., 1997; Ku et al., 1997; Leers et al., 1999; Ku and Omary, 2001). Aside from the study herein, antibodies to three proteins that specifically recognize apoptotic fragments of IF proteins have been described. One is the M30 Ab which recognizes Asp396 of human K18 in the sequence ³⁹³DALD (Leers et al., 1999). The second involves antibodies to two apoptotic fragments of glial fibrillary acidic protein (GFAP) that recognize either: (i) ²⁶³DLTD of the N-terminal GFAP fragment (located outside the L12 region) which becomes exposed after caspase cleavage, or (ii) ²⁶⁷AAARNAEC which represents the amino portion of the C-terminal fragment of GFAP that also becomes exposed after caspase digestion (Mouser et al., 2006). The third involves antibodies that recognize two apoptotic fragments of vimentin: an Ab directed to ²⁵⁶IDYD (i.e., Asp in the L12 domain) and an Ab directed to the C-terminal ²⁶⁰YSKPDC that represents the freed amino terminus of the tail-containing fragment (Nakanishi et al 2001).

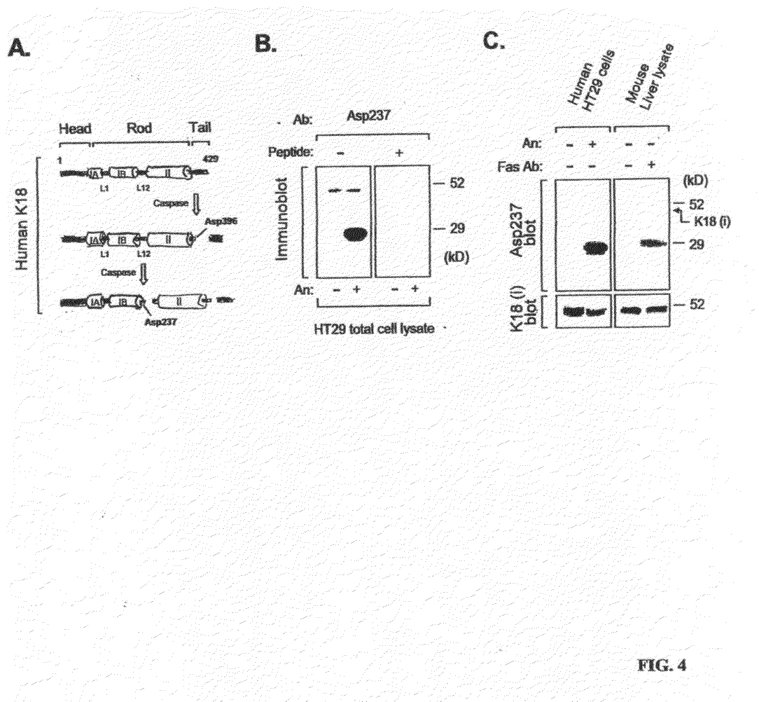

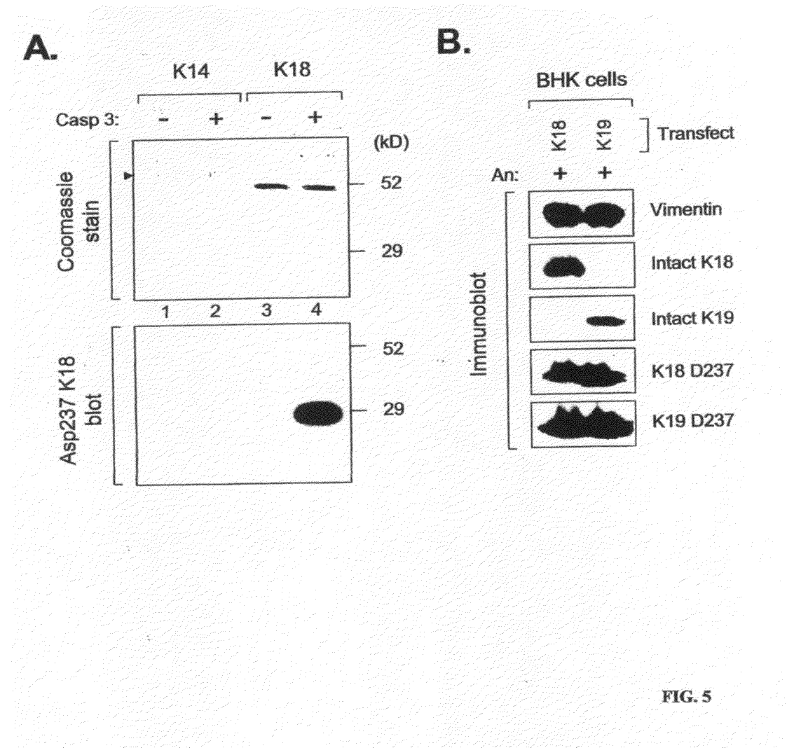

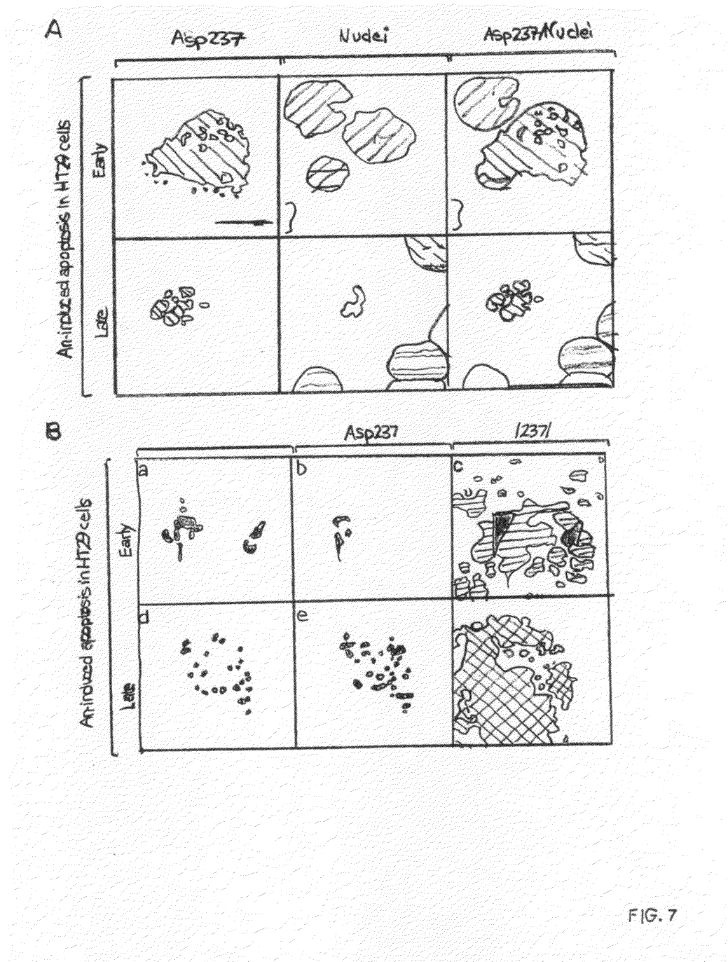

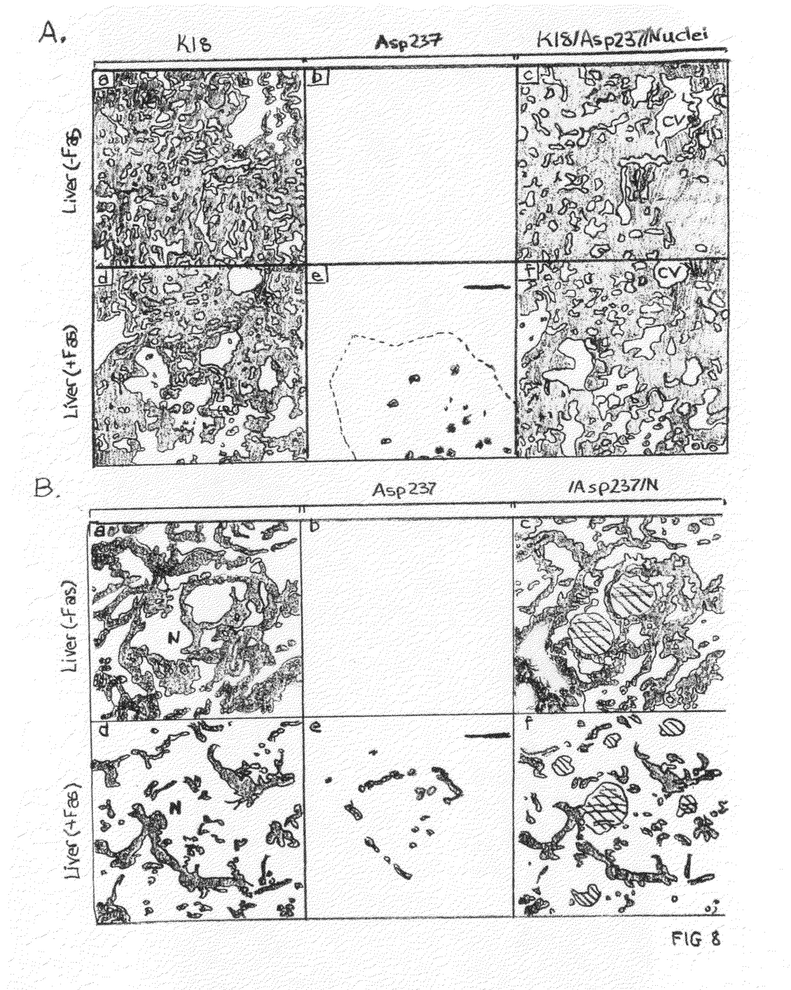

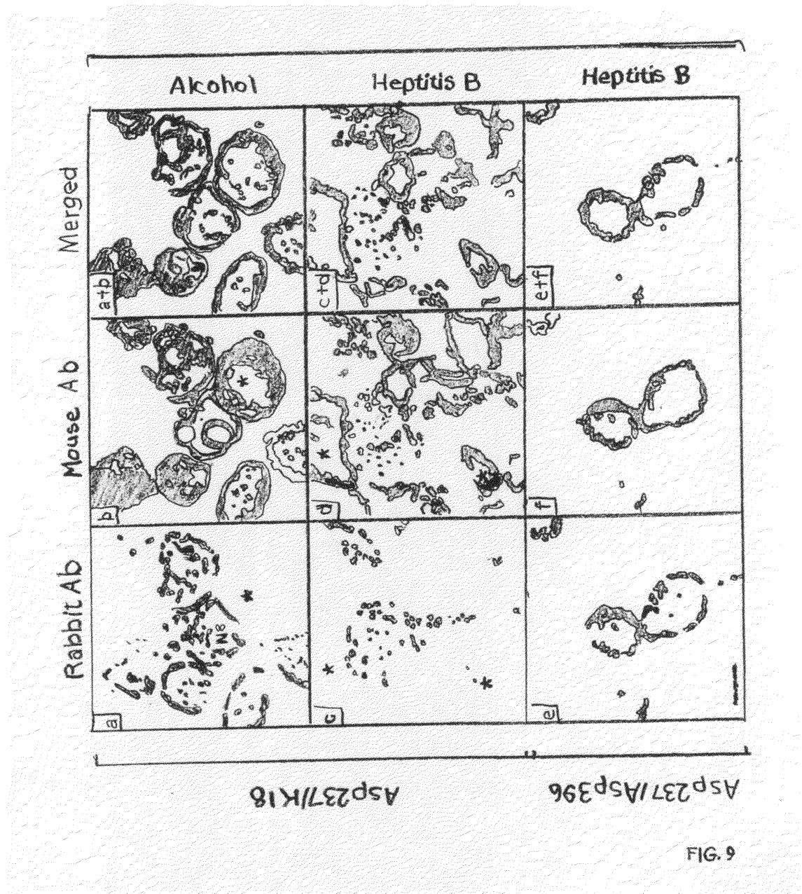

In the case of epithelial cell apoptosis, the Asp237 Ab offers important utilities and unique features as compared with the M30 antibody: (a) it recognizes both mouse and human caspase-digested K18 (FIGS. 4C, 8, 11), (b) it is highly sensitive and specific (FIGS. 4, 5) and offers broad utility of detection using a range of biochemical and histochemical methods across species (FIGS. 4, 7-11), (c) it recognizes the second sequential caspase cut site of K18 which was demonstrated directly herein by immune staining (FIG. 7). Use of the Asp237 antibody also showed that caspase cleavage of K18 is likely to occur directly on filaments which subsequently reorganize into aggregates during the late stages of apoptosis as noted in cultured HT29 cells (FIG. 7) and in vivo in mice (FIG. 8) and human (FIG. 9) tissues.

The epitope recognized by the Asp237 antibody: The dual binding to caspase-cleaved K18 and K19 by the antibodies generated to the L12 K18 and K19 Asp-containing sequences (FIG. 5B) indicates that the dominant epitope must be shared between the K18 NH₂—SSGLTVEVD²³⁷ and K19 NH₂-GGQVSVEVD²³⁷ sequences. This implicates the major common epitope as (T/S)VEVD, with the Asp-proximal valine being essential for Ab recognition. Support for this epitope assignment includes the finding that the Asp237 Ab does not recognize VEMD (FIG. 5A) (which is contained in K12-K17; see Table 1) that is generated after caspase digestion of K14 (Ku et al., 2001). Within IF proteins, the VEVD motif also occurs in K20 and lamins B1 and B2 (Table 1). Binding of the Asp237 Ab to cleaved K20, which is generated during apoptosis (Zhou et al., 2006), has not been tested but the Asp237 Ab does not recognize the lamin B1/2 caspase cleavage sequence RLVEVD (not shown) that is generated during apoptosis (Rao et al., 1996). This indicates that the environment immediately adjacent to VEVD is also important for recognition by Ab Asp237.

Interestingly, many cellular non-IF proteins harbor VEVD sequences (http://www.ncbi.nlm.nih.gov/BLAST/) yet the Asp237 Ab is highly specific for K18 and K19 and, remarkably, no other proteins are recognized by it after immunoblotting of total cell or tissue lysates (FIG. 4). This suggests that VEVD sequences within other nonkeratin proteins are not accessible to caspases during apoptosis and therefore such VEVD sequences remain masked even during apoptosis (Timmer and Salvesen, 2007). K18 Asp396, within DALD, is a unique caspase-cleavage site that is found only in the tail domain of K18 but not in other type I keratins, though DALD is conserved across species in humans and mice. Lack of convincing reactivity of the M30 mAb with mouse K18 during apoptosis is likely related to sequence differences that are immediately proximal to DALD. For example, the murine K18 sequence is GEDFSLNDALD while the corresponding human sequence is GEDFNLGDALD (underlined sequences are identical). The early cleavage at Asp396, but not Asp237, may be mediated by caspase-9 (Schutte et al., 2004; Oshima, 2002; Marceau et al., 2007 in press). During apoptosis, caspase-9 is activated in endogenous apoptotic pathways immediately after mitochondrial cytochrome c release, then caspases 3, 6, and 7 are activated which may be involved in cleaving K18/K19 Asp237 (Ku et al., 2001 and Schutte et al., 2004).

The Asp237 Ab stains many more aggregates than M30 in the late stage of apoptosis (FIG. 7B) and manifests higher biochemical detection sensitivity (FIG. 7C) which implies several scenarios. For example, the cleaved Asp237-containing fragment might be more resistant to non-caspase protease degradation as compared with the Asp396-containing C-terminal fragment in late apoptotic cells. Alternatively, the cleaved K18 amino acids 238-396 (or shorter amino-truncated peptides) may be more soluble and hence less readily detectable as aggregates by immunofluorescence staining as compared with K18 amino acids 1-237 (or shorter amino-truncated peptides).

Potential utility of the Asp237 antibody for noninvasive testing of epithelial cell apoptosis: The usefulness of K8/K18/K19 as serological markers of epithelial malignancies has been studied for more than 10 years (Rydlander et al., 1996; van Dalen et al., 1996; Barak et al., 2004). Keratin serum markers include tissue polypeptide antigen (also termed TPA) which represents total K8/K18/K19, tissue polypeptide specific antigen (termed TPS) which is derived from K18, and CYFRA 21-1 which is derived from K19. Monitoring of caspase-cleavage at K18 Asp396, using the M30 Ab, has been useful for detecting apoptosis in human epithelial tissues. For example, hepatocyte caspase activation was measured non-invasively in the blood using an M30 Ab-based ELISA as a predictor of nonalcoholic fatty liver disease in humans (Wieckowska et al., 2006) and in patients with chronic hepatitis C virus infection as a possible predictor of those who will respond to antiviral therapy (Volkmann et al., 2006). The clinical significance of determining Asp396 fragments in patient sera is valuable in the early detection of recurrence and the rapid assessment of therapy responses.

In contrast to the M30 Ab, the Asp237 Ab demonstrated a significant sensitivity for detecting changes in the second K18 caspase-generated fragment in the sera of mice injected with Fas Ab or supernatants of cultured HT29 cells. The somewhat limited sensitivity of detecting the Asp237 fragment by ELISA in the small group of tested patients as compared with sera of mice injected with Fas may be related to the extent of apoptosis in the human liver specimen, or to age of the human sera and the repeat freeze-thawing which can interfere with ELISA detection in some cases (Squotas and Tuten, 1992; Holten-Andersen et al., 2003).

The novel Asp237 antibody provides an important tool for studying K18 and K19 caspase cleavage during apoptosis in human and mouse tissues that are subjected to acute injury (e.g., Fas induced liver injury in mice) or to chronic disease (e.g., liver cirrhosis), and in the context of human cancers. The antibody also offers a tool for the non-invasive assessment of apoptosis in K18 and K19 expressing tissues.

A. Preparation of Antibodies Specific to the Cleavage at Asp237 of K18 and K19

1. Preparation of Antigens

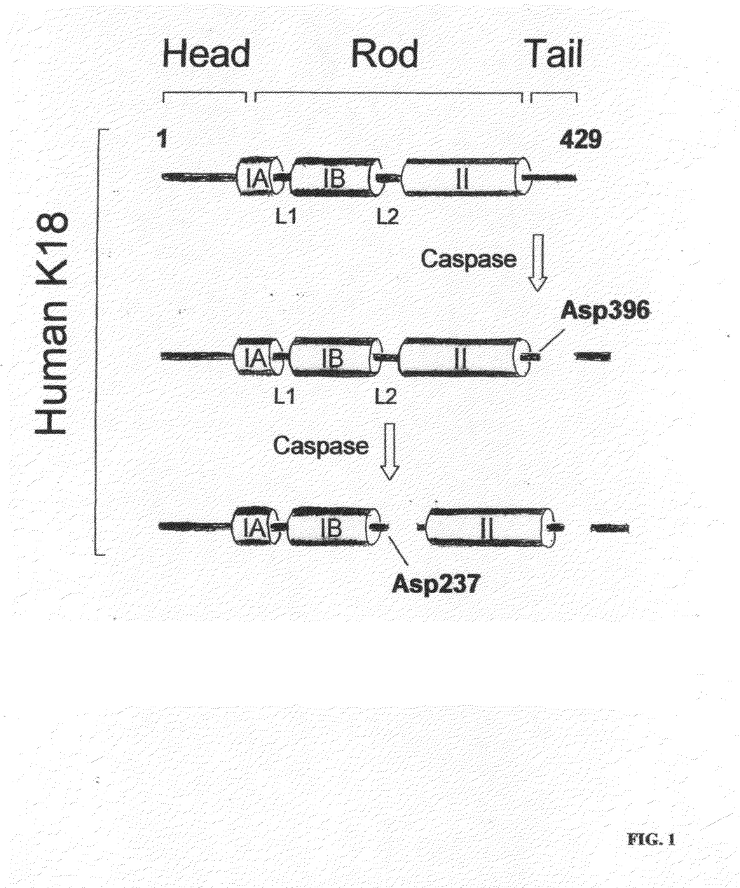

K18 and K19, on which caspases act, are intermediate filament proteins preferentially expressed in so-called “simple” (i.e., single layered) epithelial cells a found in glandular tissues such as liver, pancreas and intestine. FIG. 1 is a schematic showing caspase-cleavage sites of K18 when apoptosis is initiated by treatment with anisomycin or anti-Fas antibody. According to the present invention, the antibody can be produced against the exposed peptide that is generated after cleavage at Asp237 residue of K18 or K19. When the antibody is produced, a synthetic peptide or purified peptide fragment that has the maximum of 237 amino acid residues and contains the carboxyl terminus of caspase-cleavage site at L1-2 linker region of K18 and K19, with the last four amino acid residues of Val-Glu-Val-Asp²³⁷, can be used as an antigen. It is preferable for facilitating the coupling reaction that a Cys residue can be bound to the peptide amino terminus.

2. Generation of Polyclonal Antibodies to Cleaved Keratin 18/19

Antigens prepared as described above are administered to mammals. A wide range of animal species can be used for the production of antisera. Typically an animal used for production of antisera is a rat, a mouse, a rabbit, a sheep, a donkey, a hamster, a goat or a guinea pig. A dosage of antigens per animal is 0.1 to 100 mg when no adjuvant is used, and 10 to 1,000 μg when adjuvant is used. The adjuvants used in this invention include complete Freund's adjuvant (CFA) and incomplete Freund's adjuvant (IFA).

Immunization is principally carried out up to 10 times, preferably 2 to 5 times, at intervals of from a few days to a few weeks, preferably 2-5 week intervals. Six to 60 days after the final immunization, antibody titer is measured using conventional ELISA methods and blood is collected to obtain anti-serum when a high antibody titer is measured. Subsequently, reactivity of polyclonal antibodies in anti-serum against the cleavage product of K18/K19 is confirmed by immunoblotting, and antibodies showing strong reactivity to cleaved K18 and K19 but not to intact keratins are selected.

3. Generation of Monoclonal Antibodies to Cleaved Keratin 18/19

(1) Recovery of Antibody-Producing Cells: A synthetic peptide or purified peptide as produced above is administered as antigens to mammals, such as a rat, mouse, or rabbit. A dosage of the antigen per animal is 0.1 to 100 mg when no adjuvant is used and 1 to 100 μg when an adjuvant is used. The adjuvants include complete Freund's adjuvant (CFA) and incomplete Freund's adjuvant (IFA). Immunization is principally carried out by intravenous, subcutaneous, or intraperitoneal injection. Immunization is carried out for 1 to 10 times, preferably 2 to 5 times, at intervals of from several days to several weeks, preferably 2 to 5 week intervals. One to 60 days, preferably 1 to 14 days after the final immunization, antibody-producing cells are recovered. Examples of antibody-producing cells include spleen cells and lymph node cells.

(2) Cell Fusion: Myeloma cells to be fused with the antibody-producing cells may include generally available established cell lines of animals, such as a mouse. Preferable established cell lines used herein have drug selectivity and cannot survive in HAT selection medium containing hypoxanthine, aminopterin, and thymidine when unfused, but can survive therein only when fused with the antibody-producing cells. Next, the myeloma cells are fused with the antibody-producing cells. Briefly, 1×10⁶ to 1×10⁷ cells/mL of antibody producing cells (splenocytes) are mixed with 2×10⁵ to 2×10⁶ cells/mL of myeloma cells in an animal cell culture medium such as serum-free DMEM or RPMI-1640. Then, fusion reaction is performed in the presence of a cell fusion promoter. The cell fusion promoter (e.g., polyethylene glycol of an average molecular weight 1,000 to 6,000 daltons). Further, a commercially available cell fusion device using electric pulse stimulations (e.g., electroporation) can be used to fuse antibody-producing cells with myeloma cells.

(3) Selection and cloning of the hybridomas: Hybridomas of interest are selected from the fused cells. First, cell suspension is appropriately diluted with such as RPMI-1640 medium containing fetal calf serum, about 3×10⁵ cells/well are placed in microtiter plate wells, selection medium added to each wells, and the cells are cultured while appropriately replacing the medium. Cells growing around 14 days after the start of cultures in selection medium can be obtained as hybidomas. Next, supernatants from hybridoma cell cultures are examined by screening for the presence of antibody reacting with cleaved product K18/K19. Screening of hybridomas may be performed by any of the conventional methods. Cloning of fused cells is performed by the limiting dilution method or other established techniques. Finally, hybridomas which are cells producing monoclonal antibodies that react with cleaved fragment of K18/K19 but not with intact proteins are established.

(4) Recovery of monoclonal antibodies: Monoclonal antibodies can be recovered from established hybridomas using conventional methods, such as a cell culture method, and ascites formation method. In the cell culture method, hybridomas are grown in culture media for animal cells, such as 10% fetal calf serum-containing RPMI-1640 or MEM medium, or serum-free medium under conventional culture conditions (for example, at 37° C., under 5% CO₂) for 7 to 14 days. Then antibodies are recovered from the culture supernatants. In the ascites formation method, about 1×10⁷ cells of hybridomas are administered intraperitoneally to animals belonging to the same species of the mammals from which myeloma cells are derived so that a large number of hybridomas are grown. After 1 to 2 weeks, ascetic fluid is collected.

B. Method for Detecting Apoptosis

The antibody of the present invention may be used to detect cleaved K18 and K19 in human and animal cells, tissues or in body fluids. For example, a sample containing the cleavage product of K18/K19 may be incubated with the monoclonal antibody or polyclonal antibody of the present invention, followed by an anti-mouse or rabbit IgG antibody labeled with an enzyme such as horseradish peroxidase (HRP). The amount of the cleavage product of K18/K19 in the sample may be determined by measuring the intensity of color developed during the enzymatic reaction using a measuring device. The measured value can be used as an indicator for detecting the activity of caspase or apoptosis. A measured value greater than negative control indicates a higher activity of caspase. On the contrary, a measured value less than positive control indicates a lower activity of caspase. These measured values can be used as data for determining the progression of apoptosis.

The antibodies of the present invention have particular use as reagents in immunoassays. They include, but are not limited to, Western blotting, immunofluorescence staining, immunohistochemistry, immunoprecipitation, immuno-cytochemical staining, ELISA, dot or slot blotting, RIA, and flow cytometry.

1. Immunoassays

Immunoassays of the invention include the various types of enzyme linked immunosorbent assays (ELISAs), as are known to those of skill in the art. However, it will be readily appreciated that other useful embodiments include radioimmunoassay (RIAs) and other non-enzyme linked antibody binding assays and procedures.

The invented antibodies can be used for sandwich ELISA. The basic protocol for sandwich ELISA is as follows: A plate is coated with antibodies (called capture antibodies) specific for the antigen being assayed. The plate is then washed with a blocking agent, such as bovine serum albumin (BSA) to block non-specific binding of proteins to the test plate. The test sample such as human or animal serum is then incubated on the plate coated with the capture antibodies. The plate is then washed, incubated with said antibodies (so called detect antibodies), washed again, and incubated with a secondary antibody that is conjugated with any conventional labeling. After incubation, the unbound secondary antibody is washed from the plate. The presence of the bound secondary antibody-labeling is indicated by an appropriate detection.

In preferred embodiments, the capture antibody is an anti-K18 or anti-K19 antibody to epitope(s) between the first amino acid to Asp237 residue, and the detected antibody is the antibodies of the present invention that is immunoreactive with the neo caspase-cleaved fragment exposing Asp237 but not to the intact proteins. Said antibody of the present invention also can be used as the capture antibody. In addition, the detecting antibody may be directly labeled with a detection marker.

2. Flow Cytometry

The antibodies of the present invention may be used in methods of flow cytometry. Generally, the cells are permeabilized to allow the antibody to enter and exit the cell. After permeabilization, the cells are incubated with an antibody. It is preferred that the antibody be labeled with a fluorescent marker. If the antibody is not labeled with a fluorescent market, a second antibody that is immunoreactive with the first antibody and contains a fluorescent marker is used. After sufficient washing to insure that excess or non-bound antibodies are removed, the cells are ready for flow cytometry. Using the antibodies of the present invention for flow cytometry, apoptotic cells would be indicated by an increase in the fluorescent intensity of the cell over control, non-apoptotic cells. The apoptotic cells may be sorted by their increase in fluorescence and subjected to further analysis.

3. Western Blotting

The compositions of the present invention will find use in immunoblot analysis. Methods of Western blotting are well known to those of skill in the art and detailed methods are described (Towbin et al., 1979). Other more recent immune detection methods may be used (O'Neill et al., 2006). Generally, a protein sample is subjected to SDS-PAGE at such conditions as to yield an appropriate separation of proteins within the sample. The proteins are then transferred to a membrane (e.g., PVDF, nitrocellulose, nylon, etc.) in such a way as to maintain the relative positions of the proteins to each other. In preferred embodiments, visibly labeled proteins of known molecular weight are included within a lane of a gel. These proteins serve as a method of insuring that adequate transfer of the proteins to the membrane has occurred and as molecular weight markers for determining the relative weight of other proteins on the blot.

Subsequent to transfer of the proteins to the membrane, the membrane is submersed in blocking solution to prevent nonspecific binding of the primary antibody. In preferred embodiments, the primary antibody is said antibody to the new carboxyl terminus of K18/K19 cleaved during apoptosis. In more preferred embodiments, the antibody recognizes the new amino terminus resulted from the cleavage of K18/K19 by caspases during apoptosis.

The primary antibody may be labeled and the presence and molecular weight of the antigen may be determined by detection of the label at a specific location on the membrane. However, in preferred embodiments, the primary antibody is not labeled and the blot is further indicated with a labeled secondary antibody. This secondary antibody is immunoreactive with the primary antibody. In preferred embodiments, the secondary antibody is antibody to rabbit or mouse immunoglobulins and labeled with horseradish peroxidase or alkaline phosphatase.

4. Immunoprecipitation

The antibodies of the present invention are particularly useful for the isolation of caspase-cleaved K18/K19 fragments by immunoprecipitation. The method has been described (Ku et al, 2004). Immunoprecipitation involves the separation of the target antigen component from a complex mixture, and is useful to purify or isolate minute amounts of protein. The success of immunoprecipitation depends on the specific affinity of an antibody for its antigen as well as the strength of interaction between the antibody and Protein G or Protein A.

5. Compound Screening Assays

The present invention contemplates a process of screening compounds for their ability to affect apoptosis. Utilizing the methods and compositions of the present invention, screening assays for the testing of candidate compounds can be derived. A candidate compound is a substance which potentially can promote or inhibit apoptosis within a cell sample contacted with the substance. A screening assay of the present invention generally involves determining the ability of a candidate to affect cellular processes leading to the production of a new epitope that is immunoreactive with said antibodies.

(1) Screening Assays for Compounds that Induce Apoptosis

The present invention provides a process of determining the presence of an epitope produced in cells that are undergoing apoptosis, thus a method of detecting apoptosis. Therefore, such a method may be utilized to determine if a candidate compound is inducing apoptosis in epithelial cells. A biological sample to be screened can be a biological fluid such as extracellular or intracellular fluid or a cell or tissue extract or homogenate. A biological sample can also be an isolated cell (e.g., in culture) or a collection of cells such as in a tissue sample or histology sample.

The biological sample is exposed to the candidate compound under conditions and for a period of time sufficient for induction of apoptotic processes. Such conditions and time periods may be determined by using compounds known to induce apoptosis in a given sample. In according with a screening assay process, a biological sample that contains keratin 18 or 19 is exposed to a candidate compound being assayed. Typically, exposure is accomplished by contacting the biological sample with the candidate compound. The biological sample may be a blood sample, culture cells or a tissue sample. The tissue sample may be a biopsy, wherein the animal may not need to be sacrificed prior to collection of the sample, or may be a tissue sample collected from an animal following euthanasia or a sample collected during autopsy. The presence of apoptosis in the sample is detected by contacting the sample with the apoptosis specific antibodies of the present invention and detecting the formation and presence of antibody-polypeptide conjugates.

(2) Screening Assay for Compounds that Inhibit Apoptosis

Similar to the above assay for screening compounds for their ability to induce apoptosis, the present invention also provides methods for screening compounds that inhibit apoptosis. Generally, such methods involve subjecting a biological sample to conditions that induce apoptosis. These conditions may include, but are not limited to, pH, temperature, tonicity, or compounds capable of inducing apoptosis (e.g., anisomycin, etoposide, etc.). The candidate compounds can be added to the sample prior to, simultaneous with, or following induction of apoptosis in the biological sample. Then, the presence of apoptosis in the sample is detected by incubating the sample with the apoptosis specific antibodies of the present invention and detecting the formation and presence of antibody-polypeptide conjugates. The ability to inhibit apoptosis is indicated by a reduced level of antibody-polypeptide conjugates in the sample as compared to a control sample, subjected to the same conditions.

Experiments and Results

Reagents and antibody generation. Anisomycin (An) and monoclonal antibody (mAb) Jo2 anti-Fas were purchased from CalBiochem (San Diego, Calif., USA) and Pharmingen (San Diego, Calif., USA), respectively. In order to produce antibodies directed to K18 and K19 Asp237, the peptides NH₂-CSSGLTVEVD²³⁷ (K18) and NH₂-CGGQVSVEVD²³⁷ (K19) were synthesized using standard procedures, conjugated to keyhole limpet hemocyanin then used to immunize rabbits. The Cys, which is not part of K18/K19 sequences, was introduced in the N-terminus of the peptide to facilitate coupling to the carrier protein. The K18 and K19 sequences used for immunization are identical in mice and humans. Two rabbits were used for each peptide and the rabbit providing the highest antibody titer and specificity (not shown) is used for all the studies herein. The generated rabbit Abs were purified using beads conjugated to the two longer peptides NH₂SSGLTVEVDAPK²⁴⁰ (K18) and NH₂-GGQVSVEVDSAp²⁴⁰ (K19).

Other antibodies used in this study included: mouse mAb L2A1, which recognizes human (but not mouse) K18 and rabbit Ab 8592 which recognizes human and mouse K8/K18 (Ku et al., 2004); mAb M30 (Roche Diagnostics Corporation; Indianapolis, Ind., USA) which recognizes the 40-kd apoptotic fragment that is generated after caspase cleavage of K18 at Asp396 (Leers et al., 1999); and mouse mAb DC10, which recognizes human K18 (NeoMarkers; Fremont, Calif., USA).

Induction of apoptosis in cultured cells. Human colonic carcinoma HT29 cultured cells were obtained from the American Type Culture Collection (Manassas, Va.) and cultured as recommended by the supplier. Anisomycin (An) (dissolved in DMSO) was added (10 μg/ml final concentration) to near confluent cells and incubated for 1-48 h. Culture media were removed and saved at −80° C. for subsequent ELISA and other testing. For the preparation of cell homogenates, cells still in their culture plates were rinsed with pre-warmed PBS (37° C.) containing 5 mM EDTA then fixed for subsequent immunofluorescence staining or solubilized using 2% SDS-containing sample buffer (90° C.) followed by shearing of the DNA then analysis by SDS-PAGE and immunoblotting.

Induction of apoptosis in mouse liver. Apoptosis was induced in overnight fasted (water provided ad libitum) FVB mice by intraperitoneal injection of Fas mAb Jo2 [0.15 μg of Ab/g body weight; diluted in phosphate buffered saline (PBS) pH 7.4]. Control age and sex matched mice were injected with PBS alone. Mice were sacrificed by CO₂ inhalation 4 h after injection of the saline or Fas Ab. Blood was then collected by intracardiac puncture for ELISA testing of K18 Asp237 serum fragments. Livers were immediately resected and snap frozen in optimum cutting temperature compound for subsequent sectioning and immunofluorescence staining. All animal and human subject studies were approved by the Animal and Human Subjects Committees, respectively, at the Palo Alto VA Medical Center and Stanford University. Sera from patients with hepatitis C virus infection were collected during the year 2000 and stored at −80° C.

BHK cell transfection with keratin cDNAs. Lipofectamine 2000 (Invitrogen life technologies; San Diego, Calif., USA) was used for transient transfections of human K18 and K19 expression vectors into BHK cells, which do not express endogenous keratins). Cells were co-transfected with wild-type K8 in order to stabilize the expressed K18 or K19 via the well-established non-covalent hetero-polymerization of types I and II keratins (Ku and Omary, 2000). After 48 h, transfected cells were treated with anisomycin (10 μg/ml) for an additional 20 h to induce apoptosis, then harvested for immunoblotting.

In vitro proteolysis of keratins and other biochemical methods. Purified human K14 and K18 (kindly provided by Dr. Harald Herrmann; German Cancer Center, Heidelberg, Germany) were incubated with buffer (25 mM HEPES, 1 mM dithiothreitol, pH 7.5) alone or with buffer containing recombinant human caspase-3 at 37° C. for 3 h (Ku et al., 1997). The samples were then separated by SDS-PAGE (Laemmli, 1970) and analyzed by immunoblotting (Towbin et al., 1979). Blotted proteins were visualized using enhanced chemiluminescence as recommended by the supplier (PerkinElmer Life Sciences, Inc; Boston, Mass., USA).

Stability of the Asp237 versus the Asp396 fragments was determined by culturing HT29 cells for 6, 12, 24, 36 and 48 h in the presence of An. At the end of each incubation, floater and adherent cells were combined and pelleted (510×g for 5 min) followed by re-pelleting of the supernatant again (18,800×g for 15 min) in order to remove potential debris. SDS-containing sample buffer was then added to the supernatant (generated after the two spins) and combined cell fractions, followed by immunoblotting using the anti-Asp396 (mAb M30) and anti-Asp237 antibodies.

Immunofluorescence and immunohistochemical staining. HT29 cells were fixed in 100% methanol (−20° C., 3 min) while liver sections were acetone fixed (−20° C., 5 min). Immunofluorescence staining was carried out as described (Ku et al. 2004). Double staining utilized Texas Red-conjugated or fluorescein isothiocyanate (FITC)-conjugated second antibodies. Triple staining experiments included toto-3 iodide (Molecular Probes; Eugene, Oreg., USA) in order to visualize nuclei. Stained images were captured using a Zeiss LSM510 laser-confocal fluorescence microscope. For immunohistochemistry, formalin-fixed, paraffin-embedded mouse or human tissue sections were subjected to antigen retrieval then stained with primary rabbit antibodies followed by horseradish peroxidase-conjugated goat anti-rabbit antibodies. Human tumor/normal tissue arrays are purchased from BioChain Institute, Inc (Hayward, Calif.).

Sandwich Enzyme-Linked ImmunoSorbent Assay (ELISA) procedure. U96-Maxsorp Nunc-immuno plates (Fisher Scientific; Santa Clara, Calif., USA) were coated with purified mouse mAb DC10 (10 μg/ml) in 50 mM carbonate-bicarbonate buffer (pH 9.6) as a capture antibody and incubated overnight (4° C.). All subsequent steps were performed at room temperature with shaking. After washing, wells were blocked using 5% non-fat dry milk in PBS (1 h), followed by the addition of 50 μl of appropriately diluted cell culture medium or mouse/human serum to each well and incubating for 2 h. The culture supernatant was prepared from the media used for culturing HT29 cells grown in the presence or absence of An for 16 h (after pelleting). Wells were washed followed by the addition of the K18/K19 Asp237 Ab (1 h), rewashing, incubating with horseradish peroxidase-conjugated goat anti-rabbit IgG (1 h), then addition of the substrate tetramethylbenzidine and measuring the absorbance at 450 run using a microplate reader (Molecular Devices; Sunnyvale. CA. USA). Student's t-test was used for statistical analysis and significance was defined as a two-tailed probability of less than 0.05.

Antibody to Asp237 of K18 or K19 specifically recognizes the apoptosis-induced keratin cleavage site but not the intact proteins. Activation of caspases during apoptosis results in cleavage at conserved domains of many cellular proteins. K18 includes two established caspase digestion sites (Asp237 and Asp396) with the second site (Asp237) being conserved in both K18 and K19 (FIG. 4A). We generated rabbit anti-sera to the peptides ²²⁸SSGLTVEVD (K18) and ²²⁸GGQVSVEVD (K19) which represent the conserved D237. Analysis of the K18 Asp237 antibody by immunoblotting of lysates isolated from An-treated and untreated cells showed that the Ab recognized primarily the caspase-generated fragment (˜29 kD) in An-treated cells with slight cross-reactivity to intact K18 (˜48 kD) (FIG. 4B; two left lanes). Antibody reactivity is abolished in the presence of the peptide used for immunization (FIG. 4B, right two lanes). Identical results were obtained using the anti-K19 Asp237 antibody [not shown, HT29 cells express both K18 and K19 which undergo caspase-mediated digestion during apoptosis (Ku et al. 1997)]. Purification of the antibody using a peptide column where the peptide extends beyond the Asp237 (in order to remove intact K18 related antibodies; see Experiments and Results) removes reactivity to intact K18 as determined by immunoblotting of homogenates from HT29 cells or from mouse liver (FIG. 4C). Therefore, antibodies that are highly specific to human and mouse K18 Asp237 can be generated.

The caspase-cleavage site within the L12 linker region is conserved among most type I keratins, although the VEVD sequence seen in K18/K19 is replaced by VEMD in K12-K17 (Table 1). We asked whether the K18 Asp237 Ab recognizes the caspase-generated fragment seen in K12-K17 using purified recombinant K14 as a test substrate and K18 as a control and in vitro digestion with purified caspase-3. As shown in FIG. 5A, the K18 Asp237 Ab recognizes the caspase-3-generated K18 but not the K14 fragment. Therefore, the K18 Asp237 antibody specifically recognizes the exposed “D” in the VEVD domain but not in the VEMD that is found in the L12linker of K12-K17.

Since the K18 and K19 peptides used for immunization share four of nine amino acids (VEVD), we tested whether the K18 and K19 Asp237 antibodies are specific to the individual keratins K18 and K19 after induction of apoptosis. This was carried out by transfecting K18 and K19 separately into BHK cells which do not have endogenous keratins, followed by induction of apoptosis using An. As shown in FIG. 5B, the K18 Asp237 and K19 Asp237 antibodies recognize both caspase-cleaved K18 and K19 and as such likely share the VEVD epitope. Given this epitope sharing, all the data presented throughout the remaining studies in this document will use the anti-K18 Asp237 Ab (termed Asp237 Ab).

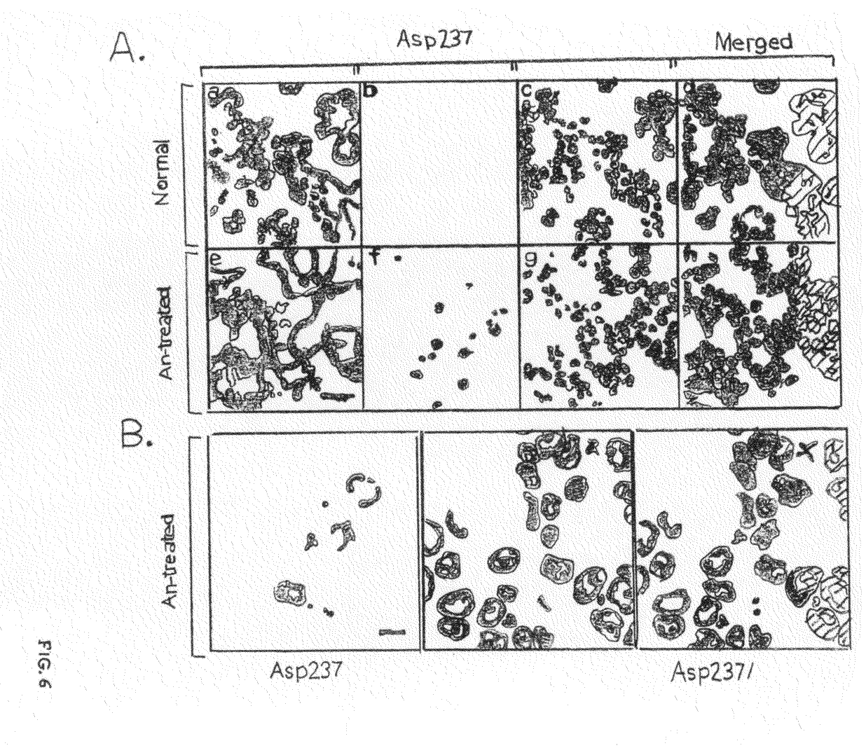

Apoptosis-induced keratin digestion is detected within filaments and aggregates. Although caspase-induced cleavage at Asp237 of K18/K19 is well described (Ku et al., 1997; Caulin et al., 1997), the topography and chronology of the process in situ in individual apoptotic cells is unclear. Hence, we used the Asp237 Ab to address these questions. As noted at lower magnification (FIG. 6A), the Asp237 Ab did not stain normal control HT29 cells (panels a-d), but stained numerous cells after treatment with anisomycin (panels e-h). Higher magnification, coupled with nuclear co-staining using Toto-3 (FIG. 6B, FIG. 7A) showed that Asp237-positive staining involved cells with condensed chromatin or fragmented nuclei consistent with apoptotic cells. Asp237 Ab staining starts 1-4 h after anisomycin treatment, with cleaved keratins being within filaments that appear to sequentially lead to reorganization into small dots (FIG. 7A, upper panel) that subsequently form large aggregates 16-20 h after exposure to the apoptotic stimulus (FIG. 7A, lower panel).

Chronology of formation and stability of the apoptotic K18 Asp237 and Asp396containing fragments. We compared caspase K18 digestion at Asp237 and Asp396 by double-staining of An-treated cells with the corresponding antibodies (FIG. 7B). At the early time point (4 h after An treatment) some cells stained only with the anti-Asp 396 Ab (e.g., FIG. 7B, cell with asterisk in panels a-c) while other cells manifested complete colocalization. During the late stage of apoptosis (16 h after An treatment) (FIG. 7B, panels d-f) digestions at Asp237 and Asp396 co-localized but there were many K18 dots that stained exclusively with anti-Asp237 while very few stained only with the Asp396 Ab. This suggests that the Asp237-containing epitope is more stable than the epitope recognized by the anti-Asp396 Ab or that the Asp396 containing species are either soluble or become released into the culture medium. This was tested further biochemically by immunoblotting HT29 cell culture supernatants or cells after treating with An for increasing intervals (0-48 h). As shown in FIG. 7C, the Asp237-containing fragment appears to be significantly more stable (based on its detectability) as compared with the Asp396-containing fragment.

Asp237 antibody detects apoptosis in mouse and human tissues as demonstrated by immunofluorescence or immunohistochemical staining. The only available antibody to Asp396 (M30) is widely used for in situ staining of K18 caspase-cleavage in tissues and cells, however it does not detect mouse K18 digestion during apoptosis (not shown). In contrast to Ab M30, the Asp237-directed Ab recognizes both human and mouse cleaved K18 as determined by immunoblotting (FIG. 4C) and immunofluorescence staining (FIG. 6-8). Asp237 Ab staining was absent in normal mouse liver (FIG. 8; panels A-b and B-b); but was readily detectable after inducing apoptosis by injection of Fas Ab. The Asp237 positive hepatocytes had short and fine filament staining when visualized under high magnification (FIG. 8; panel B-e) and positive staining in damaged areas that appear to have cell drop-off (FIG. 8; dotted area in panel A-e). The staining results are consistent with the immunoblot findings shown in FIG. 4C.

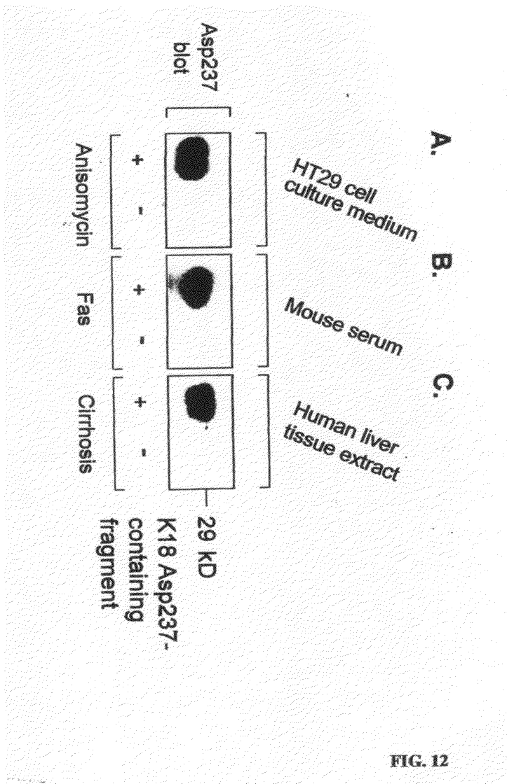

We then used double immuno-fluorescence staining to examine human cirrhotic liver explants from patients with alcohol or hepatitis B-related cirrhosis. The Asp237 Ab stained only apoptotic (filaments or tiny aggregates staining) but not other cells (e.g., cells highlighted by asterisks in FIGS. 9a and c). These cirrhotic livers with positive Asp237 staining also contained a similar 29 kD K18 fragment to that noted in FIG. 4C, which was not seen in normal human livers (FIG. 12). In contrast to mouse liver where the M30 Ab does not recognize cleaved mouse K18 efficiently, both Asp237 and M30 antibodies detect apoptosis in human diseased livers with a similar staining pattern (FIGS. 9e and f). However, there was no evidence of “early apoptosis” in these endstage liver disease tissues since M30⁺/Asp237⁻ cells were not observed in these tissues (not shown) as was noted in the staining of HT29 cells induced to undergo apoptosis (FIG. 7B).

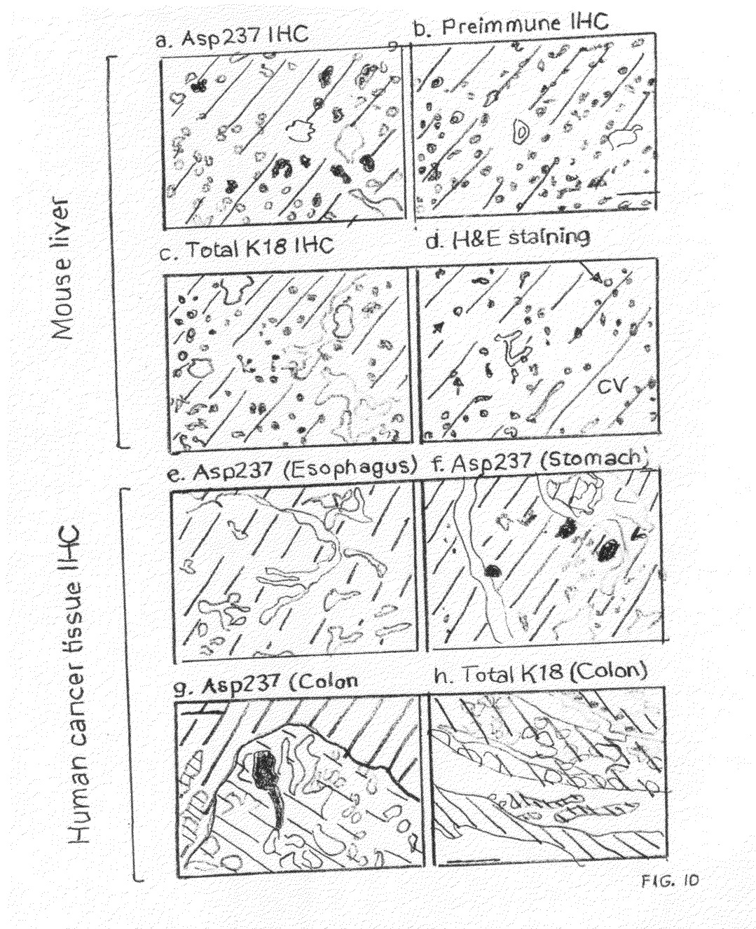

The utility of the Asp237 Ab in detecting apoptosis in formalin-fixed/paraffin embedded tissues was assessed by immunohistochemical (IHC) staining of mouse liver and several human cancer tissues. Livers from Fas-treated mice were selectively stained with the Asp237 Ab but not with pre-immune rabbit serum (FIGS. 10a and b). As another negative control, the Asp237 Ab did not stain any cells in untreated normal mouse liver (not shown). In contrast to the Asp237 Ab, a commonly utilized K18 antibody (Ku et al. Meth Cell Bio, 2004) stained all hepatocytes in the liver of Fas-treated mice (FIG. 10c). In support of the Asp237 staining, standard hematoxylin-eosin staining showed few cells with condensed or fragmented nuclei (FIG. 10d). The Asp237 Ab also detects apoptotic cells in several tested fresh formalin-fixed human adenocarcinomas including those from the esophagus, stomach and colon (FIG. 10e-h).

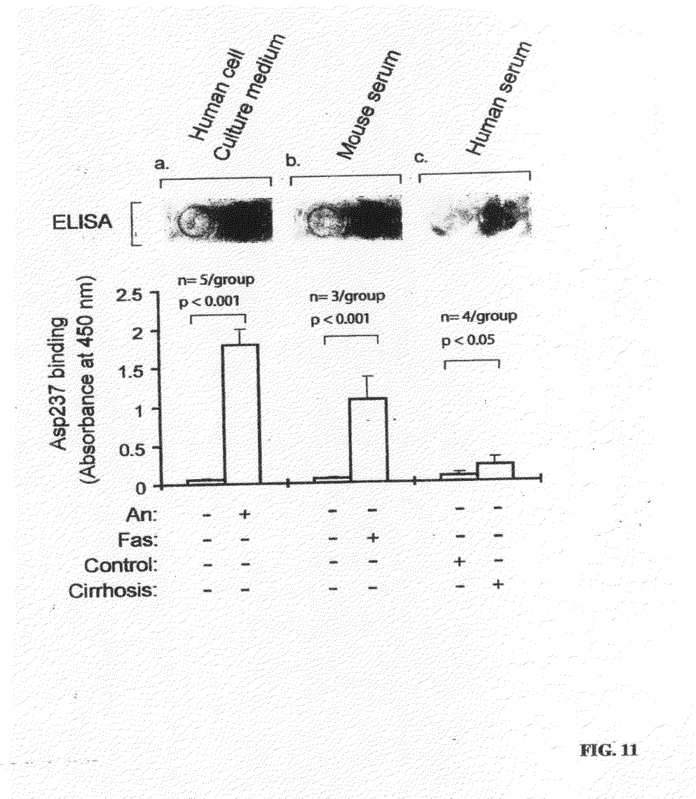

Detection of caspase-generated K18 fragments in sera of mice or patients with liver disease using the Asp237 antibody. One potential important noninvasive clinical application of antibodies directed to Asp237 is to monitor apoptosis using a sandwich ELISA assay of sera from patients with tissue-associated apoptosis. We hypothesized that Asp237 Ab will detect the presence of caspase-generated K18 fragments released into blood stream by mouse or human liver apoptotic cells. As a proof of principle, we first examined whether the K18 fragment is released by HT29 cells into the culture media during An-induced apoptosis. The cleaved K18 Asp237-containing fragment can be easily detected by ELISA (FIG. 11a) and is also detected by immune blotting (FIG. 12). The Asp237 Ab also detects the K18 apoptotic fragment in sera of mice injected with Fas antibody or in patients with cirrhosis (FIGS. 11b and c). The somewhat limited signal obtained with the human serum sample may be related to the low level of ongoing apoptosis or to the fact that the samples are archival. Presence of the cleaved K18 product was confirmed biochemically in sera of mice injected with the Fas antibody (FIG. 12). We did not have enough available human serum to confirm whether the K18 apoptotic fragment can also be detected biochemically. Taken together, these finding indicate that the Asp237 Ab is useful for noninvasive detection of cell apoptosis in cells that express K18.

Western blot analysis using the specific antibodies to detect apoptosis. Induction of apoptosis in cultured cells: Human colonic carcinoma HT29 cells (American Type Culture Collection, Manassas, Va.) were cultured in RPMI 1640 medium at 37° C., in 4% CO2 humidified incubator. Anisomycin (dissolved in DMSO) was added to the medium (10 μg/mL of final concentration) and incubated for indicated time. Culture media were removed and saved at −80° C. for ELISA. Cells grown on plates were rapidly rinsed with pre-warmed in PBS containing 5 mM EDTA, then subjected to immunofluorescence staining, or lysed using 2% SDS-containing hot sample buffer. The total cell lysates were resolved by SDS-PAGE, followed by immunoblotting using the indicated antibodies.

Apoptosis in mouse model: Apoptosis was induced after overnight fasting of age- and sex-matched normal FVB mice and then intraperitoneal injection (0.15 μg of Fas antibody/g body wt) of Fas antibody (mAb Jo2, Pharmingen, San Diego, Calif.). Mice were sacrificed by CO₂ inhalation 4 hours after injection. Livers were snap frozen in optimum cutting temperature (O.C.T.) compound for subsequent sectioning and then immunofluorescence staining. Blood samples were collected for testing K18/K19 fragments in serum by ELISA.

SDS-polyacrylamide gel electrophoresis (SDS-PAGE) and Immunoblotting: The protein samples were separated by SDS-PAGE (Laemmli, 1970) then transferred to polyvinylidene difluoride (PVDF) membranes (Millipore Corporation, Bedford, Mass.) followed by immunoblotting (Towbin et al., 1979). The antibodies used included Immunoblotted proteins were visualized using enhanced chemiluminescence as recommended by the supplier (NENTM Life Science Products, Boston, Mass.).

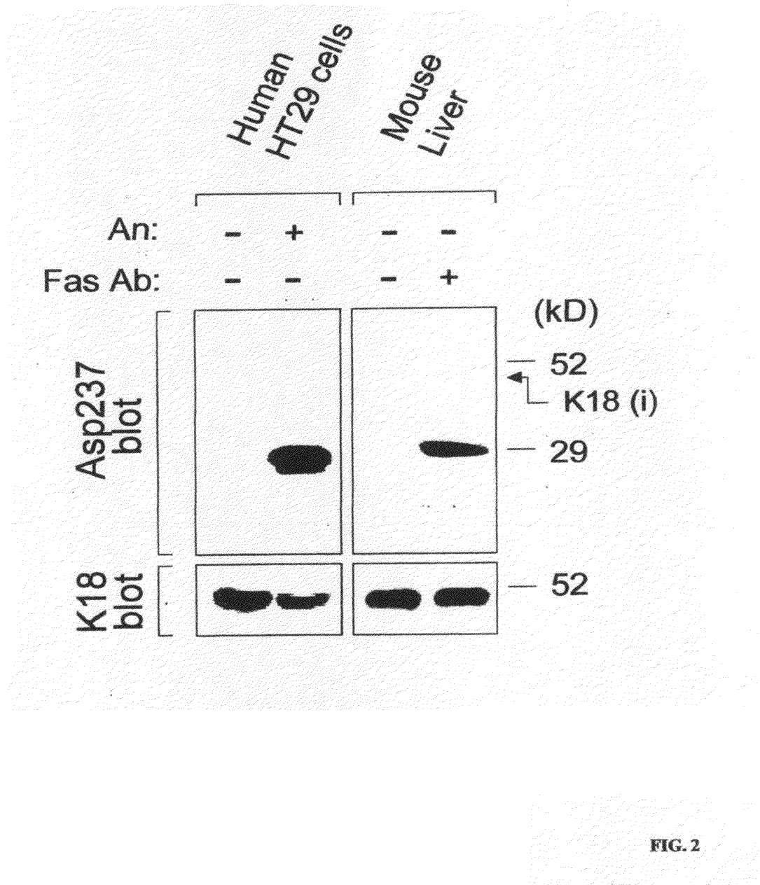

FIG. 2 shows western blots of HT29 cell lysate treated with anisomycin and liver lysates of mice injected with the anti-Fas antibody. Apoptosis was induced in HT29 cells by the addition of anisomycin (An) into culture medium, or in mouse livers by i.p. injection of Fas antibody. The cell or tissue lysates were separated on SDS-PAGE, followed by immunoblotting using Asp237 or K18 antibody. Note that the Asp237 Ab only recognizes the caspase-cleaved fragment (˜29 kDa) during apoptosis but not intact K18.

Immunofluorescence of apoptotic cells with the specific antibodies. Induction of apoptosis in culture cells: Same as described in the preceding example.

Immunofluorescence staining: HT29 cells were fixed using 100% methanol (−20° C., 3 min). The slides were blocked with PBS containing 2.5% bovine serum albumin (BSA) and 2% normal goat serum for 20 minutes then incubated with the Asp237 antibody in PBS containing 2.5% BSA (20 minutes, 22° C.). After washing, they were incubated with the blocking buffer then with Texas Red-conjugated or fluorescein isothiocyanate (FITC)-conjugated second antibodies. Triple staining experiments included toto-3 iodide (Molecular Probes, Eugene, Oreg.) for nuclear staining, after pretreatment with 0.5 mg/mL RNase A. Staining was visualized using a Zeiss LSM510 laser-confocal fluorescence microscope.

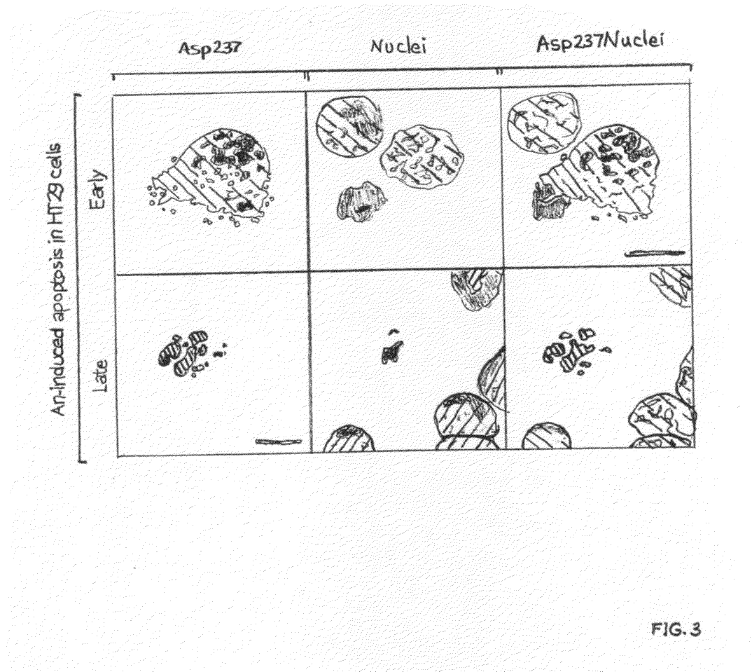

FIG. 3 shows a photograph of apoptotic cells that are fluorescently stained with the antibody. Apoptotic cells were visualized in red color using the Asp237 antibody coupled with blue nuclear staining. Upper panels show a cell undergoing apoptosis at an early stage. Note that the cell has an intact but condensed nucleus, and simultaneous positive staining on filaments and small aggregates. Lower panels show a cell residue at late stage of apoptosis with fragmented nucleus. Note the positive staining of Asp237 on the large aggregates. The antibody of the present invention was shown to react only with the cleavage product of K18/K19, but not with the intact proteins.

TABLE 1

Amino acid sequences of the conserved L12 linker
region of IF proteins. The caspase-cleavage site
has been found after the “D” residue in K18, K19,
desmin, vimentin, and lamins.

| | | |
|---|---|---|
| IFs Type | IF Protein Name | L12 Sequence |

| | | |
|---|---|---|
| I | K18 | ASSGLTVEVD |
| | K19 | VGGQVSVEVD |
| | K20 | LGNTVNVEVD |
| | K9 | NSGDVNVEIN |
| | K10 | NSGDVNVEMN |
| | K12-K17 | XXXXXXVEMD |
| II | K1-8 | XXXXXXLSMD |
| III | Desmin | QEQQVQVEMD |
| | GFAP | ARQQVHVELD |
| | Vimentin | QEQHVQIDVD |
| IV | NF-L | QYAQISVEMD |
| V | Lamin A/C | RHETRLVEID |
| | Lamin B1 | KHETRLVEVD |
| | Lamin B2 | RHERRLVEVD |

### REFERENCES

Bantel H, Lugering A, Heidemann J, Volkmann X, Poremba C, Strassburg C P, Manns M P, Schulze-OsthoffK: Detection ofapoptotic caspase activation in sera from patients with chronic HCV infection is associated with fibrotic liver injury. Hepatology 2004, 40: 1078-1087.
Barak V, Goike H, Panaretakis K W, Einarsson R: Clinical utility of cytokeratins as tumor markers. Clin Biochem 2004, 37:529-540.
Baribault H, Price J, Miyai K, Oshima R G: Mid-gestational lethality in mice lacking keratin 8. Genes Dev 1993, 7: 1191-1202.
Baribault H, Penner J, Iozzo R V, Wilson-Heiner M: Colorectal hyperplasia and inflammation in keratin 8-deficient FVBIN mice. Genes Dev 1994, 8:2964-2973.
Bredesen D E, Rao R V, Mehleni P: Cell death III the nervous system. Nature 2006, 443:796-802.
Caulin C, Salvesen G S, Oshima R G: Caspase cleavage of keratin 18 and reorganization of intermediate filaments during epithelial cell apoptosis. J Cell Biol 1997, 138:1379-1394.
Chou C F, Smith A J, Omary M B: Characterization and dynamics of O-linked glycosylation of human cytokeratin 8 and 18. J Biol Chem 1992, 267:3901-3906.
Coulombe P A, Omary M B: ‘Hard’ and ‘soft’ principles defining the structure, function and regulation of keratin intermediate filaments. Curr Opin Cell Biol 2002, 14:110122.
Coulombe P A, Wong P: Cytoplasmic intermediate filaments revealed as dynamic and multipurpose scaffolds. Nat Cell Biol 2004, 6:699-706. Review.
Dinsdale D, Lee J C, Dewson G, Cohen G M, Peter M E: Intermediate filaments control the intracellular distribution of caspases during apoptosis. Am J Pathol2004, 164:395-407.

Fuchs E and Weber K: Intermediate filaments: structure, dynamics, function, and disease. Annu Rev Biochem 1994, 63:345-382.
Fuchs E, Cleveland D W: A structural scaffolding of intermediate filaments in health and disease. Science. 1998, 279:514-519. Review.
Grassi A, Susca M, Ferri S, Gabusi E, D'Errico A, Farina G, Maccariello S, Zauli D, Bianchi F B, Ballardini G: Detection of the M30 neoepitope as a new tool to quantify liver apoptosis: timing and patterns of positivity on frozen and paraffin-embedded sections. Am J ClinPathol 2004, 121:211-219.
Habtezion A, Toivola D M, Butcher E C, Omary M B: Keratin-8-deficient mIce develop chronic spontaneous Th2 colitis amenable to antibiotic treatment. J Cell Sci 2005, 118:1971-1980.
Herrmann H, Aebi U: Intermediate filaments: molecular structure, assembly mechanism, and integration into functionally distinct intracellular Scaffolds. Annu Rev Biochem 2004, 73:749-789. Review.
Holten-Andersen M N, Schrohl A S, Brunner N, Nielsen H J, Hogdall C K, Hogdall E V: Evaluation of sample handling in relation to levels of tissue inhibitor of metalloproteinases-1 measured in blood by immunoassay. Int J Biol Markers 2003 18:170-176.
Kramer G, Schwarz S, Hagg M, Havelka A M, Linder S: Docetaxel induces apoptosis in hormone refractory prostate carcinomas during multiple treatment cycles. Br Cancer 2006, 94:1592-1598.
Ku N O, Zhou X, Toivola D M, Omary M B: The cytoskeleton of digestive epithelia in health and disease. Am J. Physiol. 1999, 277:G1108-1137. Review.
Ku N O, Liao J, Omary M B: Apoptosis generates stable fragments of human type I keratins. J Biol Chem 1997, 272:33197-33203.
Ku N O and Omary M B: Keratins turn over by ubiquitination in a phosphorylationmodulated fashion. J Cell Biol 2000, 149:547-552.
Ku N O, Omary M B: Effect of mutation and phosphorylation of type I keratins on their caspase-mediated degradation. J Biol Chem 2001, 276:26792-26798.
Ku N O, Toivola D M, Zhou Q, Tao G Z, Zhong B, Omary M B: Studying simple epithelial keratins in cells and tissues. Methods Cell BioI. 2004, 78:489-517.
Lee Je, Schickling O, Stegh A B, Oshima R G, Dinsdale D, Cohen G M, Peter M E: DEDD regulates degradation of intermediate filaments during apoptosis. J Cell BioI 2002, 158: 1051-1066.
Leers M P, Kolgen W, Bjorklund V, Bergman T, Tribbick G, Persson B, Bjorklund P, Ramaekers F C, Bjorklund B, Nap M, Jomvall H, Schutte B: Immunocytochemical detection and mapping of a cytokeratin 18 neo-epitope exposed during early apoptosis. J Pathol 1999, 187:567-572.
Laemmli U K: Cleavage of structural proteins during the assembly of the head of bacteriophage T4. Nature 1970, 227:680-685.
Liao J, Ku N O, Omary M B: ‘Stress, apoptosis, and mitosis induce phosphorylation of human keratin 8 at Ser-73 in tissues and cultured cells. J Biol Chem 1997, 272:17565-17573.
Linder S, Havelka A M, Ueno T, Shoshan M C: Determining tumor apoptosis and necrosis in patient serum using cytokeratin 18 as a biomarker. Cancer Lett. 2004, 214:1-9. Review.
Marceau N L, Schutte B, Gilbert S, Loranger A, Henfling M E R, Broers J L V, Mathew J, Ramaekers F C S: Dual Roles of Intermediate Filaments in Apoptosis. Exp Cell Res 2007 in press.
Moll R, Franke W W, Schiller D L, Geiger B and Krepler R: The catalog of human cytokeratins: patterns of expression in normal epithelia, tumors and cultured cells. Cell 1982, 31:11-24.
Mouser P E, Head E, Ha K H, Rohn I T: Caspase-mediated cleavage of glial fibrillary acidic protein within degenerating astrocytes of the Alzheimer's disease brain. Am J Pathol 2006, 168:936-9460.

Nakanishi K, Maruyama M, Shibata T, Morishima N: Identification of a caspase-9 substrate and detection of its cleavage in programmed cell death during mouse development. J Biol Chem 2001, 276:41237-41244.
Omary M B, Ku N O, Toivola D M: Keratins: guardians of the liver. Hepatology 2002, 35:251-2570 Review.
Omary M B, Coulombe P A, McLean W H: Intermediate filament proteins and their associated diseases. N Engl J Med 2004, 351:2087-2100. Review.
Omary M B, Ku N O, Tao G Z, Toivola D M, Liao J: “Heads and tails” of intermediate filament phosphorylation:” multiple sites and functional insights. Trends Biochem Sci 2006, 31:383-394.
Oshima R G: Apoptosis and keratin intermediate filaments. Cell Death Differ 2002 9:486-492.
Pallari H M, Eriksson J E: Intermediate filaments as signaling platforms. Sci STKE. 2006, 2006:pe53.
Rao L, Perez D, White E: Lamin proteolysis facilitates nuclear events during apoptosis. J Cell Biol1996, 135:1441-1455.
Rydlander L, Ziegler E, Bergman T, Schoberl E, Steiner G, Bergman A C, Zetterberg A, Marberger M, Bjorklund P, Skem T, Einarsson R, Jomvall H: Molecular characterization of a tissue-polypeptide-specific-antigen epitope and its relationship to human cytokeratin 18. Eur J Biochem 1996, 241:309-314.
Schutte B, Hentling M, Kolgen W, Bouman M, Meex S, Leers M P, Nap M, Bjorklund V, Bjorklund P, Bjorklund B, Lane E B, Omary M B, Jomvall H, Ramaekers F C: Keratin 8/18 breakdown and reorganization during apoptosis. Exp Cell Res. 2004, 297:11-26.
Schweizer J, Bowden P E, Coulombe P A, Langbein L, Lane E B, Magin T M, Maltais L, Omary M B, Parry D A, Rogers M A, Wright M W: New consensus nomenclature for mammalian keratins. J Cell Bioi 2006, 174:169-174.
Sgoutas O S, Tuten T: Effect of freezing and thawing of serum on the immunoassay oflipoprotein(a). Clin Chem 1992, 38:1873-1877.
Steinert P M, Roop D R: Molecular and cellular biology of intermediate filaments. Annu Rev Biochem. 1988, 57:593-625.
Steller H: Mechanisms and genes of cellular suicide. Science 1995, 267:1445-1449.
Tarantino G, Conca P, Coppola A, Vecchione R, Di Minno G: Serum concentrations of the tissue polypeptide specific antigen in patients suffering from non-alcoholic steatohepatitis. Eur J Clin Invest. 2007, 37:48-53.
Toivola O M, Krishnan S, Binder H J, Singh S K, Omary M B: Keratins modulate colonocyte electrolyte transport Via protein mistargeting. J. Cell Biol. 2004, 164:911-921.
Towbin H, Staehelin T and Gordon J: Electrophoretic transfer of proteins from polyacrylamide gels to nitrocellulose sheets: procedure and some applications. Proc. Natl. Acad. Sci. USA 1979, 76:4350-4354.
Timmer J C and Salvesen G S: Caspase substrates. Cell Death and Differentiation 2007, 14:66-72.
van Dalen A: Significance of cytokeratin markers TPA, TPA (cyk), TPS and CYFRA 21.1 in metastatic disease. Anticancer Res 1996, 16:2345-2349.
Volkmaml X, Comberg M, Wedemeyer H, Lehner F, Manns M P, Schulze-OsthoffK, Bantel H: Caspase activation is required for antiviral treatment response in chronic hepatitis C virus infection. Hepatology 2006, 43: 1311-1316.
Wieckowska A, Zein N N, Yerian L M, Lopez A R, McCullough A J, Feldstein A E: In vivo assessment of liver cell apoptosis as a novel biomarker of disease severity in nonalcoholic fatty liver disease. Hepatology 2006, 44:27-33.
Zhou Q, Toivola D M, Feng N, Greenberg H B, Franke W W, Omary M B: Keratin 20 helps maintain intermediate filament organization III intestinal epithelia. Mol Biol Cell 2003, 14:2959-2971.
Zhou Q, Cadrin M, Herrmarin H, Chen C H, Chalkley R J, Burlingame A L, Omary M B: Keratin 20 serine 13 phosphorylation is a stress and intestinal goblet cell marker. J BioI Chem 2006, 281:16453-16461.

1. An isolated monoclonal or polyclonal antibody that specifically recognizes caspase-generated products of K18 or K19 but does not react with intact K18 or K19, wherein the antibody reacts with the cleavage site of K18 or K19 at Asp237.

2. The antibody according to claim 1, wherein it is a polyclonal antibody.

3. The antibody according to claim 1, wherein it is a monoclonal antibody.

4. The antibody according to claim 3, wherein the monoclonal antibody binds to the epitope (T/S)VEVD of caspase cleaved K18 or K19, and wherein the Asp-proximal valine is essential for antibody recognition.

5. A method of detecting caspase activation in vitro, tissue samples or cells, wherein the method comprises the steps of: contacting one or more antibodies of claim 4 with a serum sample, a cellular sample or a tissue sample; detecting binding between the antibodies and elements from the sample.

6. A method of performing a sandwich ELISA assay, wherein the method comprises the steps of: coating a plate with one or more anti-K18 or anti-K19 antibodies to epitopes between the first amino acid to the Asp237 residue; washing the plate with a blocking agent; incubating a test sample on the plate; washing the plate and incubating it with one or more antibodies of claim 4; washing and incubating the plate with a secondary antibody that is conjugated to a labeling unit.

7. A method of performing flow cytometry, wherein the method comprises the steps of: permeabilizing one or more cells to allow entry by one or more antibodies; incubating the cells with one or more first antibodies according to claim 4; incubating the cells with a second antibody that contains a fluorescent marker and is immunoreactive to the first; washing the cells; subjecting the cells to flow cytometry, wherein an increase in fluorescence intensity of a cell over control, non-apoptotic cells indicates the presence of apoptotic cells.

8. A method of performing immunoblot analysis, wherein the method comprises the steps of: subjecting a protein sample to SDS-PAGE at such conditions to yield an appropriate separation of proteins within the sample; transferring the proteins to a membrane; submersing the membrane in a blocking solution and contacting it with one or more first antibodies according to claim 4; incubating the cells with a second antibody that contains a fluorescent marker and is immunoreactive to the first where the second antibody is conjugated to a labeling unit.

9. A method of determining whether a compound induces apoptosis in a sample, wherein the method comprises the steps of: adding one or more compounds to a sample that contains K18 or K19; adding one or more antibodies according to claim 4 to the sample; detecting binding between the antibodies and cleavage products of K18 or K19.

10. A method of determining whether a compound inhibits apoptosis in a sample, wherein the method comprises the steps of: adding one or more first compounds to a sample that contains K18 or K19; adding a second compound known to induce apoptosis in samples; adding one or more antibodies according to claim 4; detecting binding between the antibodies and the cleavage products of K18 or K19.

11. A method of detecting cancer, wherein the method comprises the steps of: contacting one or more antibodies according to claim 4 with a sample taken from a human or animal; detecting binding between the antibodies and cleavage products of K18 or K19.
